(19) United States
(12) Patent Application Publication
(10) Pub. No.: US 2026/0053927 A1
(43) Pub. Date: Feb. 26, 2026

(54) LOCALIZED RELEASE OF SYSTEMICALLY CIRCULATING THERAPEUTIC SUBSTANCES

(71) Applicant: Cochlear Limited, Macquarie University (AU)

(72) Inventors: Jonathon Kirk, Sydney (AU); Daniel Smyth, Dublin (IE); Ya Lang Enke, Baulkham Hills (AU); Wolfram Frederik Dueck, Berlin (DE)

(21) Appl. No.: 19/374,490

(22) Filed: Oct. 30, 2025

Related U.S. Application Data
(63) Continuation of application No. 17/294,521, filed on May 17, 2021.
(60) Provisional application No. 62/935,725, filed on Nov. 15, 2019.

(51) Int. Cl. A61K 41/00; A61K 9/00; A61K 9/1271; A61N 1/05

(52) U.S. Cl. CPC A61K 41/0028; A61K 9/0009; A61K 9/0046; A61K 9/0048; A61K 9/1271; A61N 1/0541

(57) ABSTRACT

Presented herein are techniques for localized release of systemically circulating therapeutic substances, which combine many of the advantages of systematic and localized administration, while eliminating many of the associated drawbacks. More specifically, in accordance with the techniques presented herein, an electro-responsive biomaterial is systemically administered to a recipient of an electrically-stimulating implantable medical device. The electro-responsive biomaterial comprises a therapeutic substance that is only activated (e.g., released) in the presence of an electromagnetic field generated by the electrically-stimulating implantable medical device.

### BACKGROUND

### FIELD OF THE INVENTION

The present invention relates generally to localized release of systemically circulating therapeutic substances within a recipient of an implantable medical device.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components

### SUMMARY

In one aspect a method is provided. The method comprises: delivering an electro-responsive biomaterial to a recipient of an implantable medical device wherein the electro-responsive biomaterial enters systemic circulation within the recipient; generating, with the implantable medical device, a localized activation field within the recipient; in response to exposure to the localized activation field, altering a physical state of the electro-responsive biomaterial to cause a therapeutic effect to the recipient. In another aspect, a kit is provided. The kit comprises reagents for generating an electro-responsive biomaterial that enters systemic circulation within the recipient, wherein the kit at least comprises a therapeutic substance and an electrically-activated carrier.

### BRIEF DESCRIPTION OF THE DRAWINGS

Embodiments of the present invention are described herein in conjunction with the accompanying drawings, in which:

FIG. 1A is a schematic diagram illustrating a cochlear implant with which certain embodiments presented herein may be implemented;

FIG. 1B is a block diagram of the cochlear implant of FIG. 1A;

FIG. 2A is a photo illustrating a perspective of a human cochlea;

FIG. 2B is a photo illustrating a top view of the human cochlear of FIG. 2A;

FIG. 3A, 3B, and 3C are schematic diagrams illustrating further details of a human cochlea and the location/positon of a stimulating assembly positioned therein, in accordance with certain embodiments presented herein;

FIG. 4 is flowchart of a method, in accordance with certain embodiments presented herein;

FIG. 5A schematically illustrates an electro-responsive biomaterial in which a conformational change occurs to activate the electrically responsive biomaterial, thereby exposing the therapeutic substance, according to aspects of the embodiments presented herein;

FIG. 5B schematically illustrates an electro-responsive biomaterial comprising a molecular switch, in which a conformational change occurs to release the contents of the pocket of the molecular switch, thereby releasing the therapeutic substance, according to aspects of the embodiments presented herein;

FIG. 5C schematically illustrates an electro-responsive biomaterial, in this case a liposome with molecules that are electro-responsive embedded in the lipid bilayer, where exposure to an electrical field causes a conformational change in the embedded molecules which generates pores in the lipid bilayer, allowing release of the therapeutic substance, according to aspects of the embodiments presented herein;

FIG. 5D schematically illustrates an electro-responsive biomaterial, in this case a liposome with molecules comprising the therapeutic substance that are covalently attached to the exterior of the shell, where exposure to an electrical field causes breakage of the covalent linkage between the liposome shell and the attached molecules, allowing the therapeutic substance to be released, according to aspects of the embodiments presented herein;

FIGS. 6A-6D are schematic diagrams illustrating the release of the therapeutic substance from an electrically-activated carrier according to the embodiments provided herein;

FIG. 7 is a schematic diagram illustrating a balance prosthesis with which certain embodiments presented herein may be implemented; and

FIG. 8 is a schematic diagram of a spinal cord stimulator with which certain embodiments presented herein may be implemented.

### DETAILED DESCRIPTION

A growing area of research and development relates to the use of pharmaceutical compounds, biological substances, bioactive substances, etc., including pharmaceutical agents/active pharmaceutical ingredients (APIs), genes, messenger RNA (mRNA) or other signalling compounds that promote recovery and resolution, chemicals, ions, drugs, etc. to treat a variety of disorders within the body of individual patient/recipient. These various substances, which are collectively and generally referred to herein as “therapeutic substances,” are delivered to induce some therapeutic results/treatment within the body of the recipient. For example, therapeutic substances may be delivered to treat ear disorders (e.g., tinnitus, hearing loss, tinnitus, Ménière's disease, etc.), to treat infections post-surgery, to fight cancer cells, to treat neurodegenerative diseases, to treat infectious diseases, etc.

There are a number of conventional approaches to the delivery of therapeutic substances within the body of a recipient. For example, certain therapeutic substances may be delivered to a recipient using a localized administration/delivery approach where the therapeutic substances are initially delivered at (i.e., close to) a target location within the recipient. With localized delivery, the therapeutic substances are delivered at the specific target location and may remain in a proximity of the target location. As such, the goal of localized delivery is that only the target location, and possibly a small amount of surrounding tissue, is exposed to the therapeutic substance. A localized delivery of a therapeutic substance may occur, for example, by inserting a catheter into the recipient. The therapeutic substance may be introduced into the body via an outlet of the catheter, which is positioned at the target location within the recipient.

In other circumstances, therapeutic substances may be delivered to a recipient using a systemic administration/delivery approach. With systemic delivery, the therapeutic substance is introduced into the circulatory system of the recipient so that, potentially, the entire body of the recipient is affected by, or at least exposed to, the therapeutic substance(s). Systemic administration of therapeutic substances can take place via, for example, enteral administration (absorption of the drug through the gastrointestinal tract) or parenteral administration (generally injection, infusion, or implantation).

Each of systematic and localized administration of therapeutic substances have advantages as well as drawbacks that may limit the use thereof for certain recipients, for certain disorders, etc. For example, as noted, the goal of localized administration is that only the target location, and possibly a small amount of surrounding tissue, is exposed to the therapeutic substance. This means that, in localized administration, the therapeutic substance must be delivered at or very close to the target location. The fact that the therapeutic substance is delivered at or very close to the target location is advantageous in that, for example, a high concentration may be administered to the target location as the rest of the recipient’s body has limited exposure to the therapeutic substance. However, a problem with localized administration is that certain areas of a recipient’s body are difficult to access in a manner that allows for the direct delivery of the therapeutic substance at that location.

In the context of the inner ear, localized administration may be difficult as the inner ear, and in particular, the apical region of a recipient’s cochlea, is difficult to access. Moreover, injecting a therapeutic substance into the cochlea may cause the loss of residual hearing (e.g., through destruction of hair cells, forming an opening the cochlea that changes the cochlear dynamics, etc.).

As noted above, a drawback of systemic administration is that, potentially, the entire body of the recipient is affected by, or at least exposed to, the therapeutic substances. As a result, systemic administered therapeutic substances must be, for example, relatively harmless to the rest of the recipient’s body, have a lower concentration that does not induce unwanted side effects outside of the target location, etc. However, systemic administration is generally easier than localized administration and has the potential for the therapeutic substances to reach anywhere within the body.

In the context of the inner ear, systemic administration may be problematic as it is difficult to deliver therapeutic substances in effective concentrations needed for the cochlea without inducing unacceptable toxic levels at other areas of the body (e.g., the pharmacokinetics limits the ability to deliver the drug to a specific location, such as high in the apex of the cochlea). That is, systemic administration of therapeutic substances to the inner ear may require such low concentrations (to prevent systemic toxicity) as to render the therapeutic substances largely ineffective for the inner ear treatment.

In view of the above, presented herein are techniques for localized release of systemically circulating therapeutic substances, which combine many of the advantages of systematic and localized administration, while eliminating many of the associated drawbacks. More specifically, in accordance with the techniques presented herein, an electro-responsive biomaterial is systemically administered to a recipient of an electrically-stimulating implantable medical device (implantable electrical stimulation device). The electro-responsive biomaterial comprises a therapeutic substance that is only activated (e.g., released) in the presence of an electromagnetic field generated by the electrically-stimulating implantable medical device. As such, although the electro-responsive biomaterial is systemically administered, the therapeutic substance only effects tissue in proximity to the implantable medical device. Accordingly, the techniques presented herein provide for “localized systemic delivery,” which can make use of systemic administration, while ensuring that only tissue in proximity to a target location is exposed to the therapeutic substance. In certain embodiments, the activation of the therapeutic substance may be an electrochemical effect (i.e., a result of the electrical field portion of the electromagnetic field). In other embodiments, the electro-responsive biomaterial may comprise a therapeutic substance activated by an electric field, a magnetic field, or both.

As used herein, an electrically-stimulating implantable medical device may be any medical device that is implanted in a recipient and, when implanted, is configured to deliver electrical stimulation (current) signals to the recipient. Examples of electrically-stimulating implantable medical devices include cochlear implants or other auditory prostheses, balance prostheses (e.g., vestibular implants), retinal or other visual prostheses, cardiac devices (e.g., implantable pacemakers, defibrillators, etc.), seizure devices, sleep apnea devices, electroporation devices, spinal cord stimulators, deep brain stimulators, motor cortex stimulators, sacral nerve stimulators, pudendal nerve stimulators, vagus/vagal nerve stimulators, trigeminal nerve stimulators, diaphragm (phrenic) pacers, pain relief stimulators, other neural, neuromuscular, or functional stimulators, etc.

As described further below, the techniques presented herein may implemented with any of a number of the above or other types of electrically-stimulating implantable medical devices. However, merely for ease of description, aspects of the techniques will be generally described with reference to a specific electrically-stimulating implantable medical device, namely a cochlear implant. Again, it is to be appreciated that the description of the techniques presented herein with reference to a cochlear implant are merely illustrative.

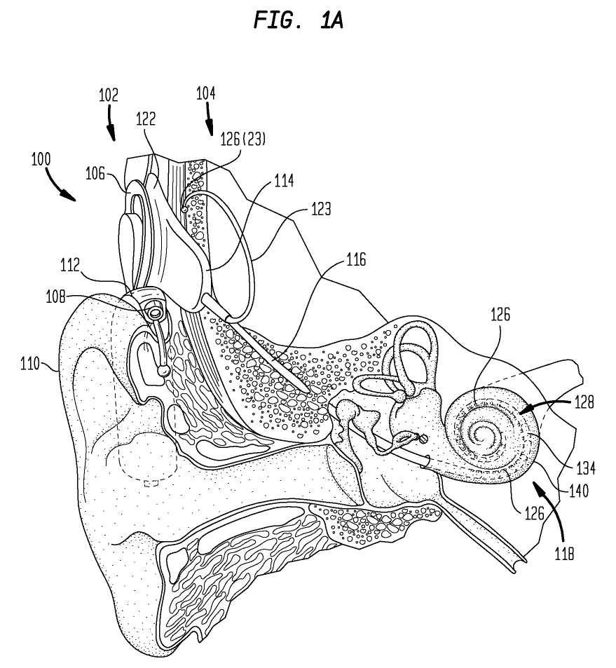

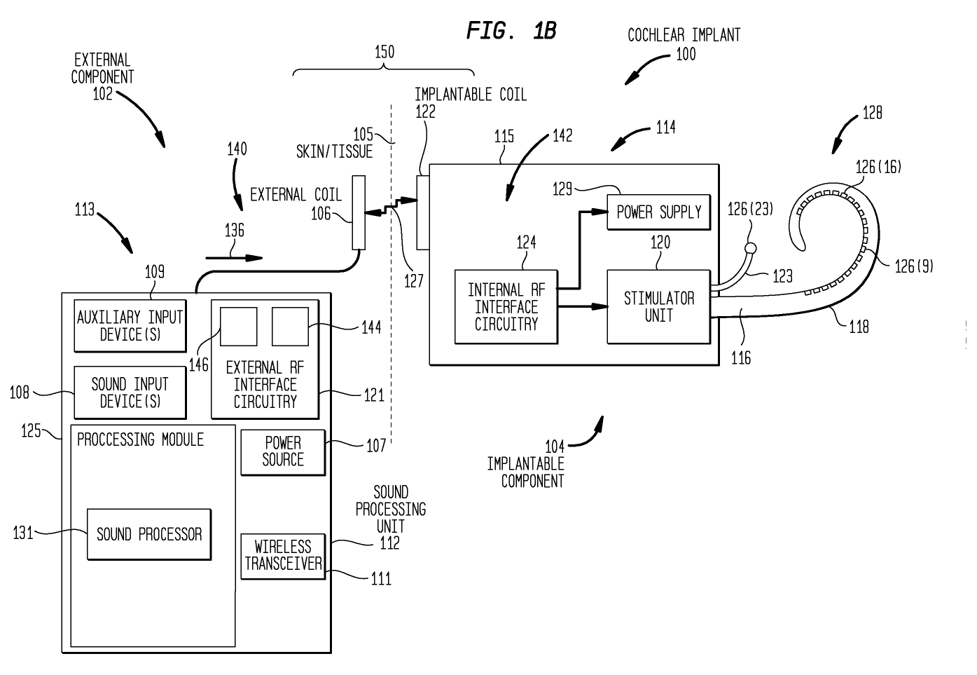

FIG. 1A is a schematic diagram of an exemplary cochlear implant 100 with which aspects presented herein may be implemented, while FIG. 1B is a block diagram of the cochlear implant 100. For ease of illustration, FIGS. 1A and 1B will be described together.

The cochlear implant 100 comprises an external component 102 and an internal/implantable component 104. The external component 102 is directly or indirectly attached to the body of the recipient and typically comprises an external coil 106 and, generally, a magnet (not shown in FIGS. 1A and 1B) fixed relative to the external coil 106. The external component 102 also comprises one or more input elements/devices 113 for receiving input signals at a sound processing unit 112. In this example, the one or more input devices 113 include sound input devices 108 (e.g., microphones positioned by auricle 110 of the recipient, telecoils, etc.) configured to capture/receive input signals, one or more auxiliary input devices 109 (e.g., audio ports, such as a Direct Audio Input (DAI), data ports, such as a Universal Serial Bus (USB) port, cable port, etc.), and a wireless transmitter/receiver (transceiver) 111, each located in, on, or near the sound processing unit 112.

The sound processing unit 112 also includes, for example, at least one battery 107, external radio-frequency (RF) interface circuitry 121, and a processing module 125. The processing module 125 may comprise a number of elements, including a sound processor 131. As described further below, the external RF interface circuitry 121 comprises data drive circuitry 144 and power drive circuitry 146 which are selectively activated/used for transcutaneous transmissions of data and power, respectively, to the implantable component 104.

In the examples of FIGS. 1A and 1B, the sound processing unit 112 is a behind-the-ear (BTE) sound processing unit configured to be attached to, and worn adjacent to, the recipient’s ear. However, it is to be appreciated that embodiments of the present invention may be implemented by sound processing units having other arrangements, such as by an off-the-ear (OTE) sound processing unit (i.e., a component having a generally cylindrical shape and which is configured to be magnetically coupled to the recipient’s head), etc., a mini or micro-BTE unit, an in-the-canal unit that is configured to be located in the recipient’s ear canal, a body-worn sound processing unit, etc.

Returning to the example embodiment of FIGS. 1A and 1B, the implantable component 104 comprises an implant body (main module) 114, a lead region 116, and an intra-cochlear stimulating assembly 118, all configured to be implanted under the skin/tissue (tissue) 105 of the recipient. The implant body 114 generally comprises a hermetically-sealed housing 115 in which internal RF interface circuitry 124, a power supply 129 (e.g., one or more implantable batteries, one or more capacitors, etc.), and a stimulator unit 120 are disposed. The stimulator unit 120 comprises, among other elements, one or more current sources on an integrated circuit (IC).

The implant body 114 also includes an internal/implantable coil 122 that is generally external to the housing 115, but which is connected to the RF interface circuitry 124 via a hermetic feedthrough (not shown in FIG. 1B). It is to be appreciated that implantable component 104 and/or the external component 102 may include other components that, for ease of illustration, have been omitted from FIGS. 1A and 1B.

As noted, the cochlear implant 100 includes the external coil 106 and the implantable coil 122. The coils 106 and 122 are typically wire antenna coils each comprised of multiple turns of electrically insulated single-strand or multi-strand platinum or gold wire. Generally, a magnet is fixed relative to each of the external coil 106 and the implantable coil 122. The magnets fixed relative to the external coil 106 and the implantable coil 122 facilitate the operational alignment of the external coil with the implantable coil.

The operational alignment of the coils 106 and 122 enables the external component 102 to transfer power (e.g., for use in powering components of the implantable component) and data (e.g., for use in generating signal signals) to the implantable component 104 via a bidirectional “transcutaneous communication link” or “closely-coupled wireless link” 127 formed between the external coil 106 with the implantable coil 122. That is, due to the operational alignment, the data drive circuitry 144 in external RF interface circuitry 121 can be used to transfer data to the implantable component 104 via the closely-coupled wireless link 127. Similarly, the operational alignment of coils 106 and 122 enables the power drive circuitry 146 to transfer power signals (power) to the implantable component 104 via the closely-coupled wireless link 127. The power signals, when received by the internal RF interface circuitry 124, may be used to power the elements of implantable component 104 and/or used to provide power to the power supply 129.

In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 1B illustrates only one example arrangement.

As noted above, sound processing unit 112 includes the processing module 125. The processing module 125 is configured to convert input audio signals into stimulation control data 136 for use in stimulating a first ear of a recipient (i.e., the processing module 125 is configured to perform sound processing on input audio signals received at the sound processing unit 112). Stated differently, the sound processor 131 (e.g., one or more processing elements implementing firmware, software, etc.) is configured to convert the captured input audio signals into stimulation control data 136 that represents stimulation signals for delivery to the recipient. The input audio signals that are processed and converted into stimulation control data may be audio signals received via the sound input devices 108, signals received via the auxiliary input devices 109, and/or signals received via the wireless transceiver 111.

In the embodiment of FIG. 1B, the stimulation control data 136 is provided to the external RF interface circuitry 121, where the data drive circuitry 144 transcutaneously transfers the stimulation control data 136 (e.g., in an encoded manner) to the implantable component 104 via external coil 106 and implantable coil 122. That is, the stimulation control data 136 is sent by the data drive circuitry 144 over the closely-coupled wireless link 127. The internal RF interface circuitry 124 is configured to receive the stimulation control data 136 via implantable coil 122 and to provide that data to the stimulator unit 120. The stimulator unit 120 is configured to utilize the stimulation control data 136 to generate electrical stimulation signals (e.g., current signals) for delivery to the recipient’s cochlea via the stimulating assembly 118. In this way, cochlear implant 100 electrically stimulates the recipient’s auditory nerve cells, bypassing absent or defective hair cells that normally transduce acoustic vibrations into neural activity, in a manner that causes the recipient to perceive one or more components of the input audio signals.

More specifically, as noted above, stimulating assembly 118 is configured to be at least partially implanted in the recipient’s cochlea 140. Stimulating assembly 118 includes a plurality of longitudinally spaced intra-cochlear electrical contacts (electrode contacts or electrodes) 126 that collectively form an electrode contact array 128 configured to, for example, deliver electrical stimulation (current) signals generated based on the stimulation control data 136 to the recipient’s cochlea. In certain examples, the electrode contacts 126 may also be used to sink stimulation signals from the recipient’s cochlea.

FIG. 1A illustrates a specific arrangement in which stimulating assembly 118 comprises twenty-two (22) intra-cochlear electrode contacts 126, labeled as electrode contacts 126(1) through 126(22). It is to be appreciated that embodiments presented herein may be implemented in alternative arrangements having different numbers of intra-cochlear electrode contacts.

As shown, the intra-cochlear electrode contacts 126(1)-126(22) are disposed in an elongate carrier member 134. The carrier member 134 has a center longitudinal axis and an outer surface. The carrier member 134 is formed from a non-conductive (insulating) material, such as silicone or other elastomer polymer. As such, the carrier member 134 electrically isolates the intra-cochlear electrode contacts 126(1)-126(22) from one another. As shown in FIG. 1B, the intra-cochlear electrode contacts 126(1)-126(22) are each spaced from one another by sections/segments of the carrier member 134.

The stimulating assembly 118 extends through an opening in the recipient’s cochlea (e.g., cochleostomy, the round window, etc.) and has a proximal end connected to stimulator unit 120 via lead region 116 and a hermetic feedthrough (not shown in FIG. 1B). Carrier member 134 and lead region 116 each includes a plurality of conductors (wires) extending there through that electrically connect the electrode contacts 126 to the stimulator unit 120.

Also shown in FIG. 1A is an extra-cochlear electrode contact 126(23). The extra-cochlear electrode contact 126(23) is an electrical contact that is configured to, for example, deliver electrical stimulation to the recipient’s cochlea and/or to sink current from the recipient’s cochlea. The extra-cochlear electrode contact 126(23) is connected to a reference lead 123 that includes one or more conductors that electrically couple the extra-cochlear electrode contact 126(23) to the stimulator unit 120. It would be appreciated that the example use of one extra-cochlear electrode contact 126(23) is merely illustrative and that embodiments presented herein may be used with other electrode combinations (e.g., more or less external electrodes).

FIGS. 1A and 1B illustrate an arrangement in which the cochlear implant 100 includes an external component (e.g., sound processing unit 112). However, it is to be appreciated that certain embodiments of the present invention may be implemented in cochlear implants having alternative arrangements. For example, embodiments of the present invention may be implemented in a totally implantable cochlear implant (or other totally implantable medical device). A totally or fully implantable medical device, such as a totally implantable cochlear implant, is a device in which all components of the device are configured to be implanted under skin/tissue of a recipient. Because all components are implantable, the totally implantable medical device operates, for at least a finite period of time, without the need of an external device.

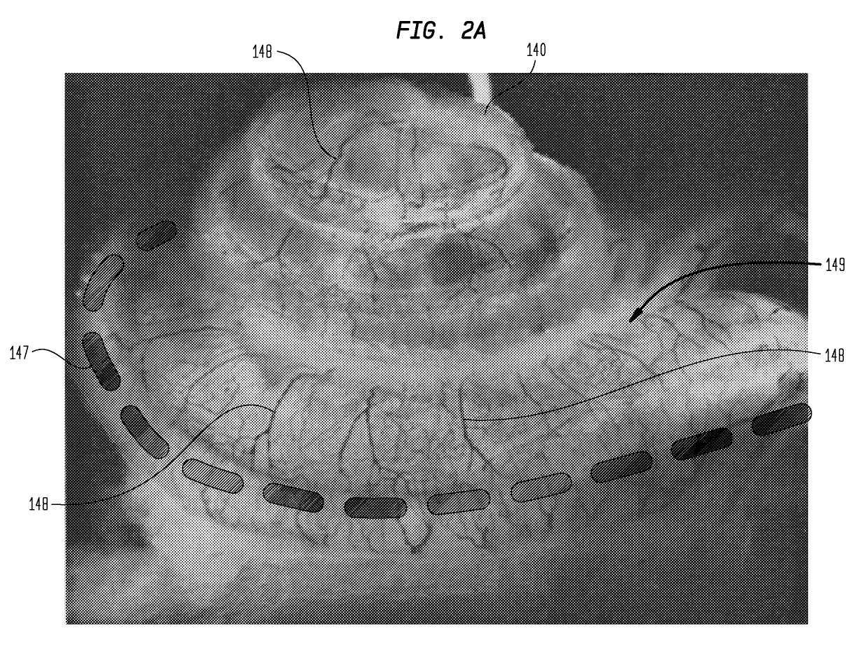

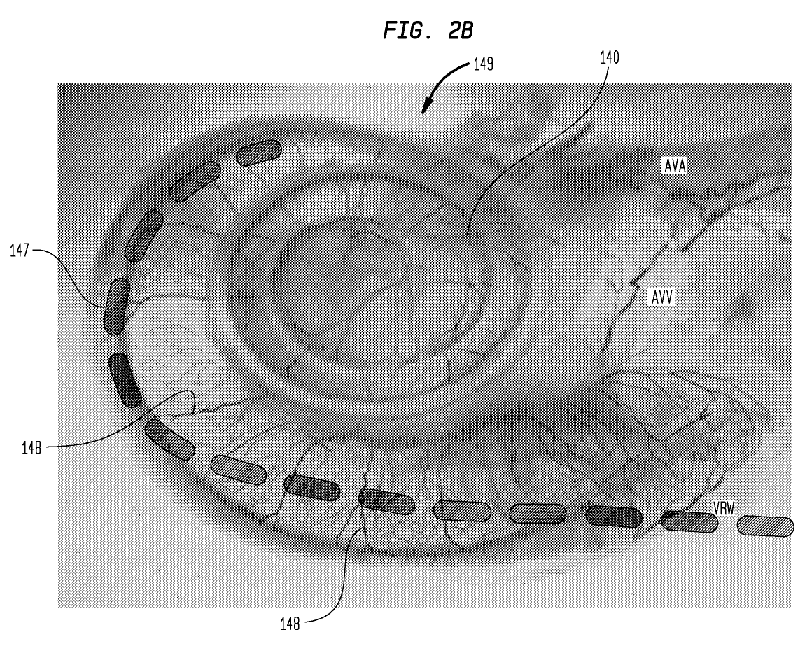

FIG. 2A is a photo illustrating a perspective of cochlea 140, while FIG. 2B is a photo illustrating a top view of the cochlea 140. The photos of FIGS. 2A and 2B have both been annotated to show the location/path 147 of stimulating assembly 118 within the cochlea 140.

FIGS. 2A and 2B also illustrate the outer wall 149 of the cochlea 140. As shown, there are numerous blood vessels 148 within the outer wall 149 of the cochlea 140. These blood vessels 148 provide a significant vascular supply to the tissue of the cochlea 140.

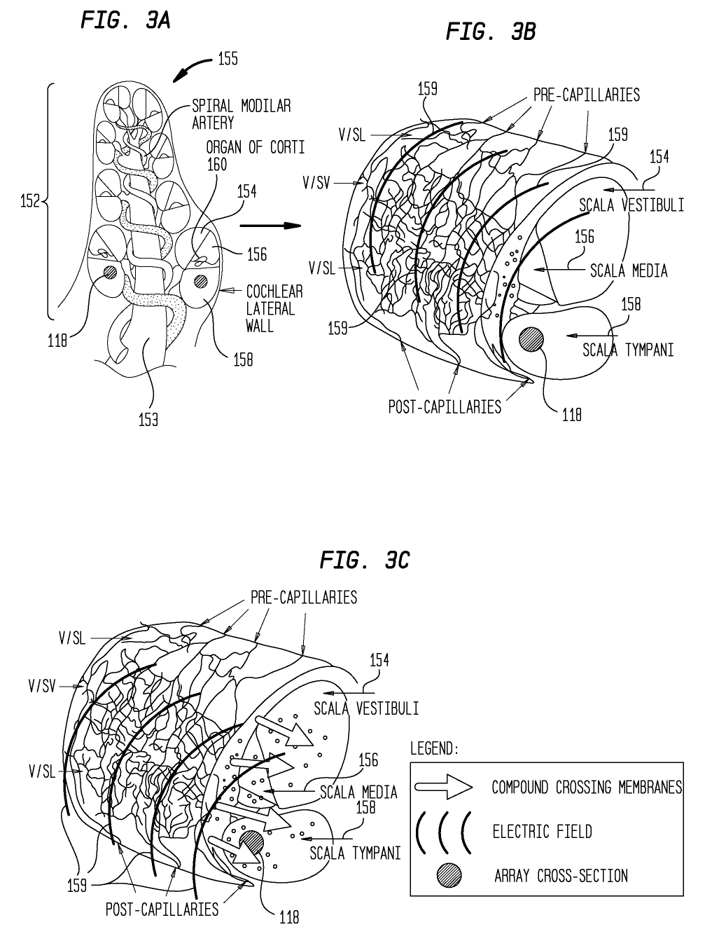

In addition, FIGS. 3A, 3B, and 3C are schematic diagrams illustrating further details of cochlea 140, as well as the location/positon of stimulating assembly 118 therein. More specifically, FIG. 3A is cross-sectional view of the cochlea 140 partially cut-away to display the canals of the cochlea 140, while FIGS. 3B and 3C are cross-sectional perspective views of one turn of the canals of the cochlea 140. For ease of description, FIGS. 3A-3C will be described together.

Cochlea 140 is a conical spiral structure comprising three parallel fluid-filled canals or ducts, collectively and generally referred to herein as canals 152. Canals 152 comprise the tympanic canal 158, also referred to as the scala tympani 158, the vestibular canal 154, also referred to as the scala vestibuli 154, and the median canal 156, also referred to as the scala media 156. Cochlea 140 spirals about modiolus 153 several times and terminates at cochlea apex 155. The organ of Corti 160 is situated on the basilar membrane in the scala media 156 and contains rows of 16,000-20,000 hair cells (not shown) which protrude from its surface. In the examples of FIGS. 3A-3C, the stimulating assembly 118 of cochlear implant 100 spirals around the modiolus 153 within the scala tympani 158.

Also shown in FIGS. 3A-3C is the outer wall 149 of the cochlea 140. As noted above with reference to FIGS. 2A and 2B, the outer wall 149 includes numerous blood vessels 148 which provide a substantial vascular supply to the tissue of the cochlea 140.

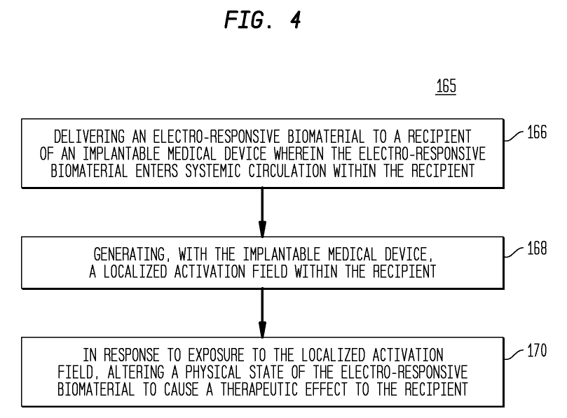

FIG. 4 is a flowchart of a method 165 for localized release of systemically circulating therapeutic substances, in accordance with embodiments presented herein. For ease of description and understanding, method 165 will be described with reference to cochlear implant 100 and cochlea 140, as detailed with reference to FIGS. 2A-2B and 3A-3C.

Method 165 begins at 166 wherein an electro-responsive biomaterial is delivered to a recipient of an implantable medical device such that the electro-responsive biomaterial enters systemic circulation within the recipient (i.e., an electro-responsive biomaterial is systemically administered to the body of a recipient of cochlear implant 100). At 168, the cochlear implant 100 generates a localized activation field within the body of the recipient, specifically within the cochlea 140. At 170, in response to exposure to the localized activation field, a physical state of the electro-responsive biomaterial is altered to cause a therapeutic effect to the recipient. Further details regarding the operations at 166, 168, and 170 are provided below.

More specifically, referring first to the operations of 166, the electro-responsive biomaterial may be delivered (systemically administered) to the body of the recipient in a number of different manners. In certain embodiments, the electro-responsive biomaterial may be delivered to the body of the recipient via enteral administration, where the electro-responsive biomaterial enters the recipient’s circulatory system via absorption through the gastrointestinal tract. In certain circumstances, enteral administration may be divided into three different categories, depending on the entrance point into the gastrointestinal tract, namely: oral (by mouth), gastric (through the stomach), and rectal (from the rectum). As such, methods of enteral administration that may be used to introduce the electro-responsive biomaterial to the body of the recipient include, for example, oral (by mouth) administration (e.g., a pill, tablet, capsule, solution, softgel, suspension, emulsion, syrup, elixir, tincture, hydrogel), sublingual administration (i.e., dissolving the electro-responsive compound under the tongue), an/or rectal administration (e.g., ointment, suppository, enema, murphy drip, nutrient enema. Gastric introduction may utilize a tube through the nasal passage (e.g., nasogastric tube) or a tube through the skin leading directly to the stomach (e.g., percutaneous endoscopic gastrostomy tube), etc.

In certain embodiments, the electro-responsive biomaterial may be delivered to the body of the recipient via parenteral administration. As used herein, parenteral administration refers to any routes of administration that do not involve absorption via the gastrointestinal tract, including intravenous (IV) (into a vein), intramuscular (IM) (into a muscle), subcutaneous (SC or SQ) (under the skin), transdermal, (onto the skin) nasal (via the nasal passage), ocular (via the eye), etc. Intravenous administration includes delivery of the electro-responsive biomaterial directly into the circulatory system (i.e., directly into systemic circulation), either by direct injection or infusion via a peripheral or central vein. Intramuscular and subcutaneous administration generally include injection of the electro-responsive biomaterial in a manner that establishes a deposit or “depot” of the electro-responsive biomaterial that will be released gradually into the systemic circulation.

In summary, the techniques presented herein use one of enteral administration and/or parenteral administration to systemically introduce the electro-responsive biomaterial into the body of the recipient. The results of the enteral administration and/or parenteral administration is that the electro-responsive biomaterial enters into systemic circulation in the body of the recipient.

Referring next to the operations at 168, the cochlear implant 100 generates a localized activation field within the body of the recipient. More specifically, the cochlear implant 100 is implanted within the cochlea 140 of the recipient and is configured to deliver electrical stimulation (current) signals to the cochlea 140 of the recipient. That is, the cochlear implant 100 sources (delivers) current to the recipient via one or more implanted electrode contacts 126(1)-126(23), while also sinking the current via a different one or more of the implanted electrode contacts 126(1)-126(23). The electrode contact(s) sourcing the current to the recipient at a given time may be referred to as the “source electrode contact(s)” or “source electrode(s)” while the electrode contact(s) sinking the current at a given time may be referred to as the “sink electrode contacts” or “sink electrodes.” As a result, current flows from the source electrode(s) to the sink electrode(s) through fluid and/or tissue of the recipient.

The flow of current generated by the cochlear implant 100 induces a localized electromagnetic field (EMF) within the immediate vicinity/proximity of cochlea 140. That is, the electromagnetic field is referred to as being “localized” because the electromagnetic field is only induced in close proximity to the area of current flow (i.e., in the fluid and tissue in proximity to the flowing current). In general, an electromagnetic field is a physical field produced by moving electrically charged objects (current) and can be viewed as the combination of an electric field and a magnetic field. The electric field is produced by stationary charges, while the magnetic field is produced by moving charges (current). The way in which charges and currents interact with the electromagnetic field is described by Maxwell's equations and the Lorentz force law.

As noted above, the electro-responsive biomaterial is systemically administered to the recipient, meaning the electro-responsive biomaterial is introduced into the circulatory system of the recipient (i.e., the electro-responsive biomaterial enters into systemic circulation within the body of the recipient). Systemic circulation provides functional blood supply to all body tissue of the recipient, while picking up carbon dioxide and waste products. That is, systemic circulation carries oxygenated blood from the left ventricle, through the arteries, to the capillaries in the tissues of the body (i.e., supplies oxygen and nutrients to the body cells). From the tissue capillaries, the deoxygenated blood returns to the right atrium of the heart through a system of veins.

Since the electro-responsive biomaterial enters systemic circulation, the electro-responsive biomaterial will also flow with the oxygenated blood to tissues of the body and from the tissue capillaries back to the recipient’s heart. Stated differently, the electro-responsive biomaterial is able to circulate throughout the recipient’s body, including into the blood vessels 148 in the outer wall 149 of the cochlea 140. At this location (e.g., when the electro-responsive biomaterial flows through and/or near the blood vessels 148), the electro-responsive biomaterial is exposed to the localized electromagnetic field generated by the cochlear implant 100.

In accordance with embodiments presented herein, the electro-responsive biomaterial comprises a therapeutic substance and an electrically-activated carrying vehicle (electrically- activated carrier). As described further below, the electrically-activated carrier renders the therapeutic substance inactive/inert in the absence of a “localized activation field.” However, at 170 of FIG. 4, when the electro-responsive biomaterial enters a localized activation field (i.e., in response to exposure of the electro-responsive biomaterial to the localized activation field), the electrically-activated carrier reacts in a manner that enables the therapeutic substance to activate and become bioavailable (e.g., release from its carrier, attain an activated or exposed state, etc.) to cause a therapeutic effect for the recipient at tissue in proximity to the localized activation field. That is, in response to exposure to the localized activation field, a physical state of the electro-responsive biomaterial is altered to cause a therapeutic effect to the recipient. Also as described further below, the reaction of the electrically-activated carrier within the localized activation field may be different for different types of electro-responsive biomaterials with different types of electrically-activated carriers.

In accordance with embodiments presented herein, the localized activation field that activates the therapeutic substance to cause a therapeutic effect at the tissue of the recipient is the electric field and/or the magnetic field generated by the cochlear implant 100 (i.e., the electric field and/or the magnetic field components of the localized electromagnetic field within the immediate vicinity/proximity of cochlea 140 generated by the cochlear implant 100). Stated differently, the delivery of the electrical stimulation (current) to the cochlea 140 of the recipient generates the localized activation field. Therefore, when the electro-responsive biomaterial flows through and/or near the blood vessels 148, the electro-responsive biomaterial is exposed to localized activation field which then activates the therapeutic substance for delivery to tissue of recipient, including to the cochlea 140.

In each of FIGS. 3B and 3C, the localized activation field generated by cochlear implant 100 is represented by lines 159. In FIG. 3C, arrows 161 illustrate therapeutic substances crossing the membranes to enter the cochlea 140 from the blood vessels 148. For example, in certain embodiments the change induced/caused by the localized activation field enables the therapeutic substance to cross the blood labyrinth barrier and enter the cochlea 140 from the systemic route. This may enable more effective coverage of the cochlea 140 than other conventional technologies and methods.

In some aspects, an electro-responsive biomaterial may include one or more chemical and/or biological molecules, and may comprise a single type of molecule or multiple types of molecules. In certain embodiments, the electrically-activated carrier may be formed from one or more small molecules or proteins (e.g., the electro-responsive biomaterial comprises a therapeutic substance bound to one or more proteins). In other embodiments, the electrically-activated carrier may be formed from one or more nanoparticles/supramolecular structures (e.g., the electro-responsive biomaterial comprises a therapeutic substance bound to one or more nanoparticles/supramolecular structures. These examples are described in further detail below.

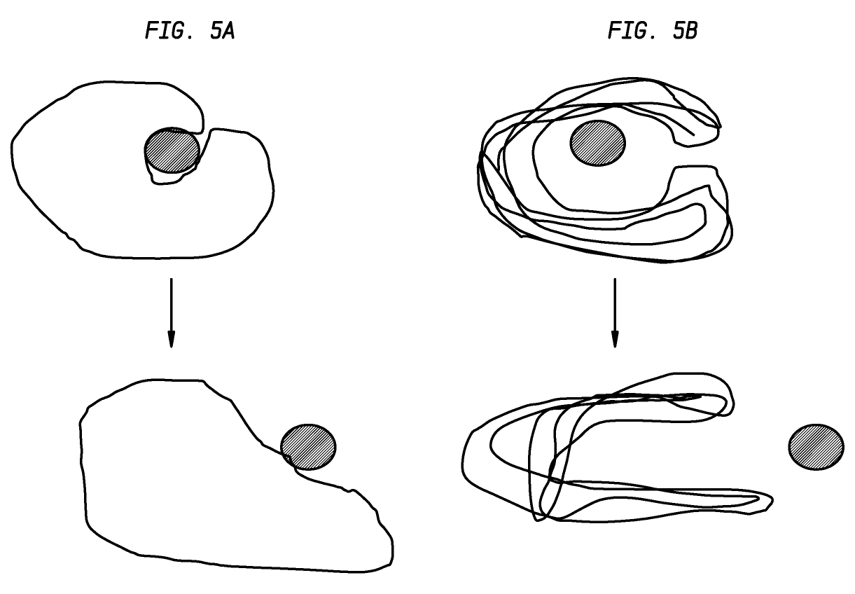

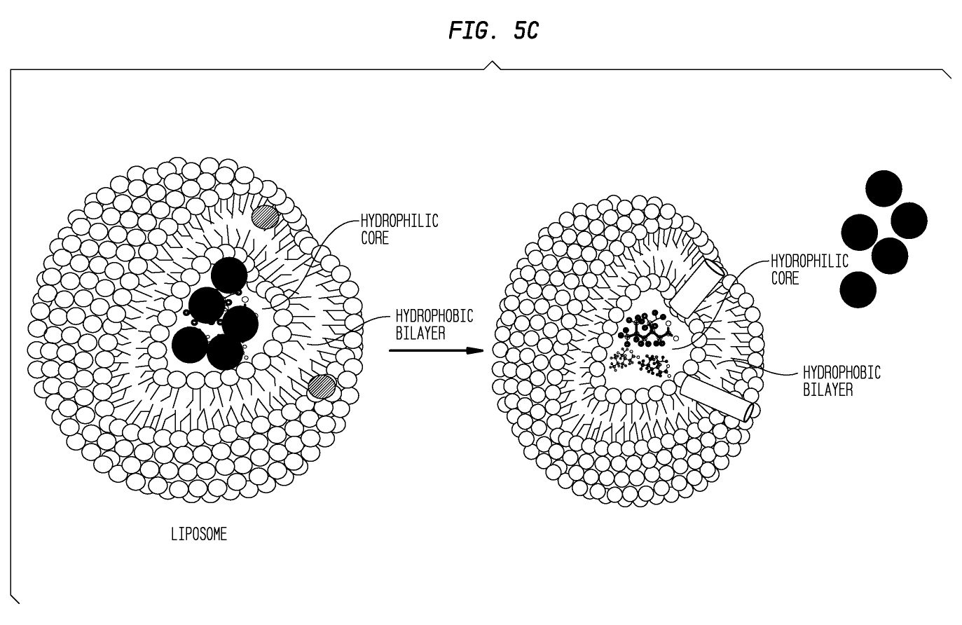

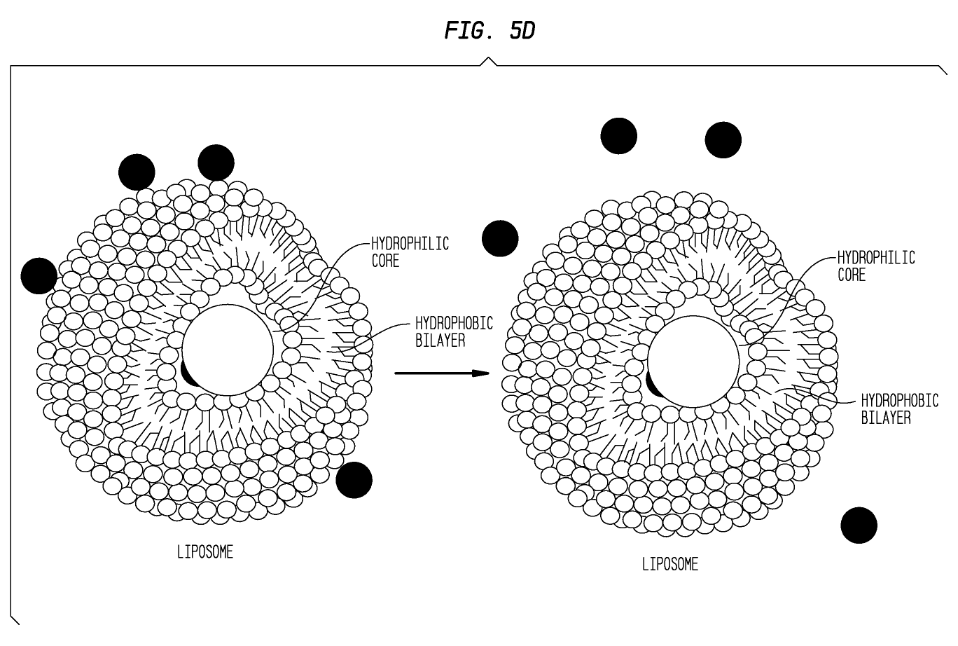

In some aspects, the biomaterial may comprise a single type of molecule. In this case, the biomaterial may be delivered to the patient in an inactive state, and may undergo a conformational change to an active state (e.g., to a therapeutically active molecule) in the presence of an electrical field, as shown in FIG. 5A. In this example, the therapeutic substance and the electrically-activated carrier is contained in the single molecule. For example, a small molecule or protein may undergo a conformational change to expose an active site upon exposure to an electrical field. In this example, the molecule has at least two conformations, e.g., a first conformation in which the active site is not accessible and a second conformation in which the active site is accessible. In some aspects, the electrical field may change a physical property of the molecule (e.g., pH, kinetic energy, net electric charge) to induce a conformational change of the molecule. As examples, the electro-responsive biomaterial may comprise a small molecule, a peptide, a protein, an antibody, etc. or any other suitable type of molecule.

The electro-responsive biomaterial may be organic or inorganic and may be configured for host-guest interactions which may involve structures referred to as molecular tweezers. In other aspects, the biomaterial may comprise a molecular switch, such that when a conformational change is produced in response to external electrical stimuli (e.g., electric field), the electro-responsive biomaterial is converted from an inactive to an active state.

In other aspects, the electro-responsive biomaterial comprises a therapeutic substance and an electrically-activated carrier. In this example, the therapeutic substance and the electrically-activated carrier are separate molecules. In accordance with embodiments herein the therapeutic substance and the electrically-activated carrier can each have a number of different forms. In this representation, the therapeutic substance and the electrically-activated carrier are not covalently linked to one another. In some aspects, the electrically-activated carrier may comprise a molecule having a cleft, pocket, or cavity, into which the therapeutic substance may be placed. Upon exposure to the electric field, the electrically-activated carrier may undergo a conformational change to release the therapeutic substance at the site of the electric field as shown in FIG. 5B. In this example, the electrically-activated carrier may be a small molecule, a protein, a polymer, a peptide, or any other type of molecule capable of forming a cleft, pocket or cavity into which a therapeutic substance may be situated.

Examples of these types of electrically-activated carriers may include but are not limited to, molecular switches or molecular tweezers (e.g., an organic molecule that forms a pocket capable of complexing with a therapeutic substance through non-covalent interactions, such as hydrogen bonding, hydrophobic or van der Waals forces, aromatic stacking or metal coordination, etc.) (see, FIG. 5B). These molecules may undergo a conformational change to release the therapeutic substance upon exposure to an electric field.

In other aspects, the electrically-activated carrier may include but is not limited to a molecule capable of forming a layer which encapsulates the therapeutic substance. In this example, the therapeutic substance and the electrically-activated carrier are not covalently linked to one another. For example, the molecule may form a supramolecular assembly (e.g., a liposome (lipid bilayer), a micelle (lipid monolayer), a membrane, a nanoshell, an organic nanoparticle, an inorganic nanoparticle, a dendrimer, a protein, a fliposome, etc. or any other material that may be used to encapsulate the therapeutic substance).

In some aspects, the supramolecular assemblies are sensitized or otherwise configured to change conformation when exposed to a localized activation field (e.g., electric field) of certain attributes. The supramolecular assemblies may be organic or inorganic and may form shells or layers into which molecular tweezers or switches or other molecules are embedded. The embedded molecules may undergo conformational changes in response to specific electrical conditions, generating an opening in the shell/layer, and releasing the contents of the interior of the shell, as shown in FIG. 5C.

In other aspects, the therapeutic substance may be attached as shown in FIG. 5D to the supramolecular assembly (e.g., interior or exterior of the supramolecular assembly) via direct linking or linking via a crosslinker, etc. The attachment may depend, for example, on attributes of the therapeutic substance (e.g., physical and chemical properties). In these embodiments, the therapeutic substance can be released from the nanoparticle by breaking the bond (e.g., oxidation) when passing through a localized activation field (e.g., the localized activation field causes the supramolecular assembly to release the therapeutic substance, e.g., by breaking the covalent bond). In some aspects, the therapeutic substance may be attached through a covalent linkage to the exterior of the supramolecular assembly, while in other aspects, the therapeutic substance may be attached through a covalent linkage to the interior of the supramolecular assembly. In either case, activation causes release of the therapeutic substance.

In still other aspects, fliposomes may be generated, such that exposure to a stimuli (e.g., pH, metal complexation, electric field, temperature, radiation, light, etc.) causes a conformational change which disrupts the lipid layer, leading to release of the therapeutic substance in the interior of the shell.

A therapeutic substance may include one or more molecules capable of exhibiting a therapeutic effect when administered to a patient. Therapeutic substances may include small molecules and biologics including but not limited to a peptide, a polypeptide, an antibody, a nucleic acid, a mRNA, a CRISPR/Cas9 complex, a lipid, a steroid, a carbohydrate, a proteoglycan, and analogs, derivatives, mixtures, fusions, combinations or conjugates thereof.

In another embodiment, the therapeutic substance is a nucleic acid, selected from the group consisting of: an antisense molecule (RNAi), an aptamer, a cDNA, a gene or gene fragment (e.g., for gene therapy, optionally provided with the CRISPR/Cas9 complex, CRISPR/Cas9 prime editing), an oligonucleotide, a regulatory sequence, a ribozyme, a triple-helix forming molecule, including analogs, derivatives, and combinations thereof.

Still other examples of therapeutic substances include but are not limited to: antibiotics, anti-inflammatory drugs, angiogenic or vasoactive agents, growth factors, cytotoxic agents (e.g., tumour suppressers), and biologics.

Therapeutic substances may also include nucleic acid sequences. Nucleic acid sequences according to the techniques provided herein include those encoding enzymes, ligands, receptors, regulatory factors, and structural proteins that may be administered to a patient to treat a condition. Therapeutic nucleic acid sequences also include sequences encoding cytoplasmic proteins, mitochondrial proteins, nuclear proteins, plasmalemma-associated proteins, secreted proteins, serum proteins, bacterial antigens, parasitic antigens, protozoal antigens and viral antigens to treat a condition or elicit a therapeutic response.

Proteins or polypeptides which can be expressed by therapeutic nucleic acid sequences according to the techniques provided herein include antibodies, apolipoproteins, bacterial antigens, drugs, enzymes, immunoglobulins, neurotransmitters, oncogenes, parasitic antigens receptors, structural proteins, toxins, tumour antigens, tumour suppressers, and viral antigens.

In operation, electrical stimulation and electric fields can lead to localized changes to attributes of the molecules (e.g., pH, temperature, redox reactions, temperature, etc.), which can induce localized redox reactions or generate local cations which could prompt disassembly of the electro-responsive biomaterial (i.e., to separate the therapeutic substance and the electrically-activated carrier). In certain examples, a conformational change of one or more of the units comprising the supramolecular assembly occurs in response to this external stimuli (e.g., electric field) allowing the therapeutic substance to be released. For example, molecular switches may undergo a conformational change to release contents within a binding pocket. A shell or layer may contain molecules which change conformation to allow the interior contents to be released. Further, if the electro-responsive biomaterial is sufficiently polarized, exposure to a directional electric field could may disrupt an organized structure/layer of the electrically-activated carrier, allowing release of an encapsulated therapeutic substance. In general, the electrical stimulation (e.g., electrical field) could interact with the electrically-activated carriers in any of a number of different manners, leading to local release of the therapeutic substance or activation of the electro-responsive biomaterial.

In accordance with embodiments presented herein, the electrically-activated carrier renders the therapeutic substance inactive/inert in the absence of a localized activation field (i.e., electro-responsive biomaterial remains stable and in systemic circulation until exposed to a localized activation field). However, when the electro-responsive biomaterial is exposed to a localized activation field, the electrically-activated carrier releases the therapeutic substance. In accordance with embodiments presented herein, the localized activation field may have any of a number of different attributes to cause the electrically-activated carrier to release the therapeutic substance.

For example, in certain embodiments the molecular structure of the electrically-activated carrier could be tied to (selected to correspond to) specific field attributes (e.g., one or more of a specific magnitude, a specific voltage, a specific polarity, etc.) that cause the electrically-activated carrier to release the therapeutic substance. In such embodiments, the electrically-activated carrier will only release the therapeutic substance when the electro-responsive biomaterial is exposed to a localized activation field having these specific attribute(s).

In other embodiments, the molecular structure could be field-agnostic such that the electrically-activated carrier will release the therapeutic substance in a broader number of fields, such as any localized electromagnetic field having a voltage above a certain (threshold) voltage.

In certain embodiments the localized activation field is generated by an implantable medical device that is used only for the localized release of the systemically circulating therapeutic substance. However, in other embodiments, the localized activation field is generated by an implantable medical device that is also used to stimulate the tissue of the recipient for a separate therapeutic effect (e.g., to induce a hearing percept at a cochlea of the recipient). There are a number of different types of such partially or fully implantable medical devices with/in which embodiments presented herein may be implemented. For example, the techniques presented herein may be implemented with cochlear implants or other auditory prostheses, such as auditory brainstem stimulators, electro-acoustic hearing prostheses, direct cochlear stimulators, bimodal hearing prostheses, etc. The techniques presented herein may also be used with balance prostheses (e.g., vestibular implants), retinal or other visual prosthesis/stimulators, occipital cortex implants, sensor systems, cardiac devices (e.g., implantable pacemakers, defibrillators, etc.), drug delivery systems, catheters, seizure devices (e.g., devices for monitoring and/or treating epileptic events), sleep apnea devices, electroporation devices, spinal cord stimulators, deep brain stimulators, motor cortex stimulators, sacral nerve stimulators, pudendal nerve stimulators, vagus/vagal nerve stimulators, trigeminal nerve stimulators, diaphragm (phrenic) pacers, pain relief stimulators, other neural, neuromuscular, or functional stimulators, etc.

Release or activation of the therapeutic substance may occur in any suitable manner, including changing the magnitude of the electric field, pulsing the electric field, varying the area of the electric field, etc. Thus, the electric field may be controlled to attain a desired local concentration of the therapeutic substance and to maintain this concentration or control aspects of delayed or extended release of the therapeutic substance near the release site. The electric field may be configured to cause release of the therapeutic substance in a therapeutically effective amount to treat a condition or disorder or disease.

In some aspects, a kit is provided, wherein the kit comprises reagents for generating an electro-responsive biomaterial (e.g., reagents to couple the therapeutic substance to an electrically-activated carrier according to the embodiments provided herein. For example, the kits may include molecular switches having pockets of a known size into which a therapeutic may fit. In other aspects, kits for forming nanoshells, liposomes or other supramolecular assemblies may be provided. For example, the therapeutic may mixed with a physiologically compatible buffer and electrically-activated carrier, to form supramolecular assemblies for delivery. In other embodiments, the kit may comprise a pharmaceutical composition comprising a delivery vehicle and a physiologically compatible buffer.

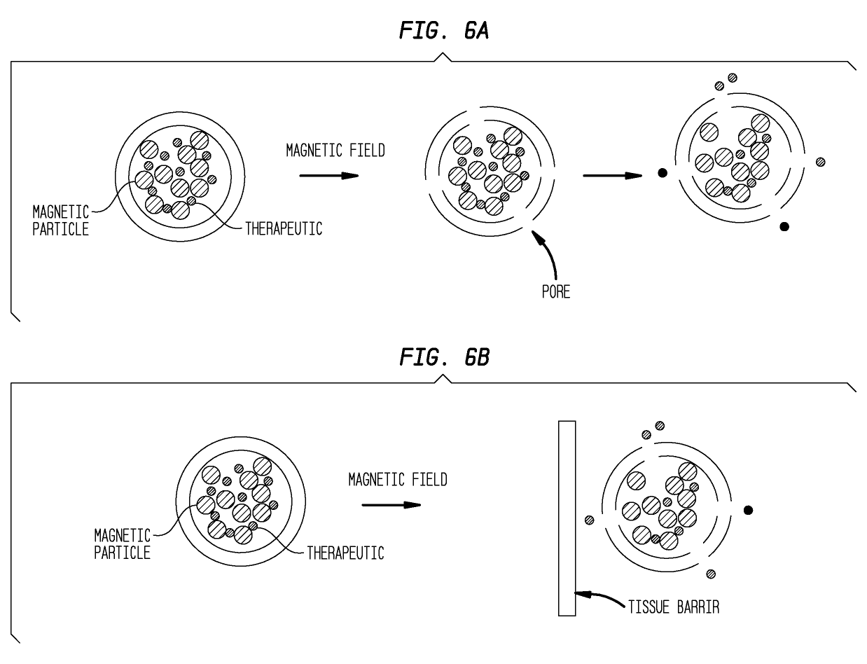

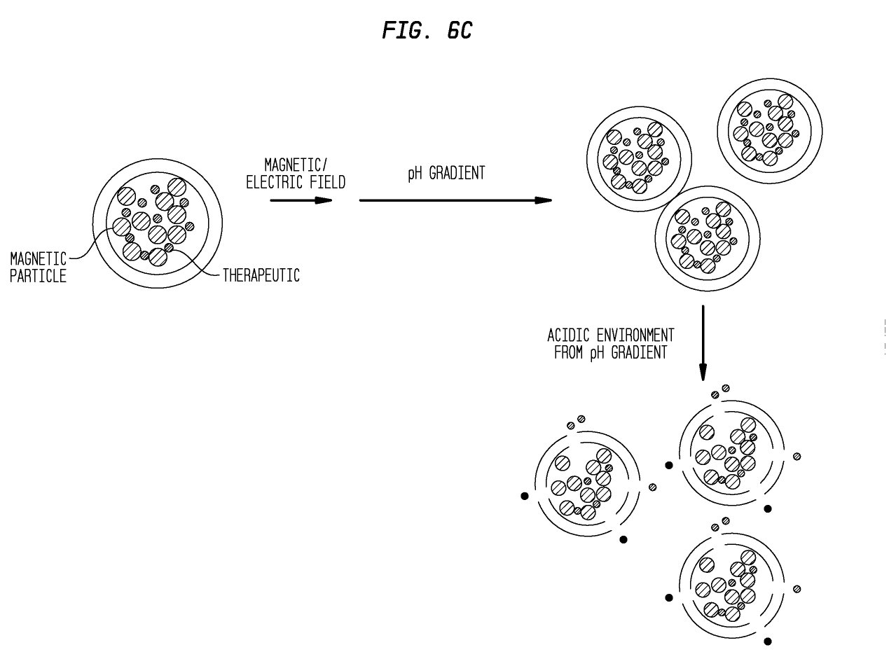

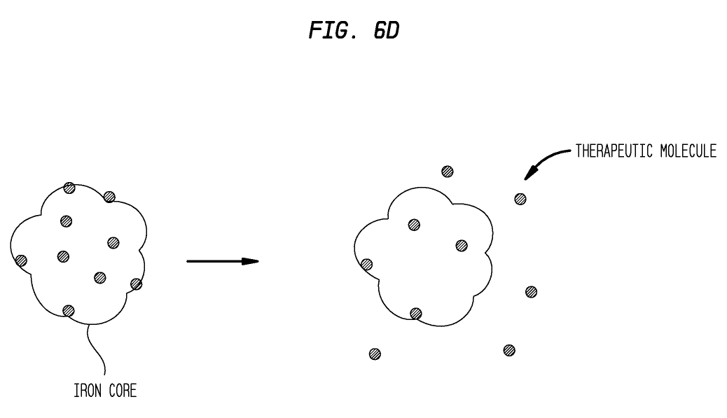

FIGS. 6A-6D are schematic diagrams illustrating the release of the therapeutic substance from an electrically-activated carrier according to various embodiments provided herein. In FIG. 6A, a magnetic field may be applied to biomaterial comprising magnetic iron nanoparticles, such as superparamagnetic iron oxide nanoparticles, that are encapsulated by a liposome with a lipid bilayer. The electric field/magnetic field may heat up the nanoparticles, increasing thermal energy and leading to a more fluid lipid bilayer, thereby increasing the porosity of the lipid bilayer. Thus, the magnetic field may cause formation of pores in the liposome membrane, leading to release of therapeutic contents through pores allowing delivery of the encapsulated therapeutic at the target site.

In other aspects, iron oxide may be additionally incorporated into the lipid bilayer as well as within the interior of the liposome, to further facilitate pore formation. In other aspects, the liposome may be formulated to be a temperature sensitive liposome or polymer, such that heat from an electric/magnetic field leads to dissolution of the liposome, releasing the contents of the liposome due to an increase in thermal energy. In some aspects, the temperature sensitive liposome or polymer may conduct electricity to generate heat.

In still other aspects, the electric field/magnetic field may facilitate accumulation of the magnetic particles in the vicinity of the tumor site, e.g., the magnetic particles may accumulate in the presence of a localized magnetic field/electric field.

This example is not intended to be limited to the particular structures shown in FIG. 6A, and is intended to cover a variety of structures, e.g., a temperature sensitive liposome with a therapeutic core, a liposome with a therapeutic core and magnetic particles in the lipid bilayer, a liposome with a therapeutic core containing magnetic particle, or any combination thereof.

FIG. 6B shows another embodiment, in which a liposome containing a therapeutic core and magnetic particles migrates in an electric field/magnetic field generated by the implantable device. In some aspects, the implantable device may be configured to cause the liposome to directionally migrate to the site of a tumor, or even into the tumor tissue itself, due to the polarized or magnetic properties of the liposome. In further aspects, this approach may be used to target polarized molecules, not necessarily attached to a magnetic particle, to a tumor site.

In FIG. 6C, an electric field is applied to a physiological location to generate a pH gradient. The electro-responsive biomaterial, in an acidic environment from the pH gradient, becomes activated due to the change in pH and releases the therapeutic at the target location. In this example, a liposome may be formulated to become more porous in an acidic environment. In FIG. 6D, an iron nano-particle comprising a magnetic Fe₃O₄ core with a shell of aqueous stable polyethylene glycol (PEG) conjugated to a biomaterial is shown. This type of particle may be targeted to specific locations in response to application of a magnetic field. The therapeutic may be released once reaching a particular location. For example, the pH of tumor-associated tissue or inflammatory tissue may be decreased as compared to normal tissue. Accordingly, the magnetic field may be used to accumulate liposomes in a particular location, and once reaching the tumor or inflammatory environment, reduced pH may cause release of the biomaterial.

In still other aspects, therapeutic materials may be embedded in polymers that are sensitive to an electric or magnetic field. For example, sulfonate polystyrenes, poly(thiophene)s and poly(ethyloxazoline) may each be sensitive to electric fields, and the embedded materials may be released from the polymeric scaffold in response to changes in electric current. In some aspects, such polymers may contain a high concentration of ionisable groups, e.g., along the backbone of the polymeric chain. For example, the electric current may cause a change in pH which leads to a disruption in the 3-D shape of the polymer. Ideally, the electric field will be provided at a strength to facilitate drug release, without stimulating nearby nerve endings in surrounding tissue. Additional examples of electro-responsive materials may include chitosan, alginate, or hyalouronic acid. Synthetic polymers that are electro-responsive may include allyl amine, vinyl alcohol, acrylonitrile, methacrylic acid, and vinylacrylic acid. In other aspects, combinations of synthetic and naturally occurring polymers may be used. In some aspects, the polymers may be polyelectrolytes. In still other aspects, biomaterials may include both polymers that are not electro-responsive with polymers that are electro-responsive.

The devices provided herein are operable with any suitable therapeutic delivery system, e.g., polymers, liposomes, gels, biologic molecules, small molecules, etc., responsive to an electric field.

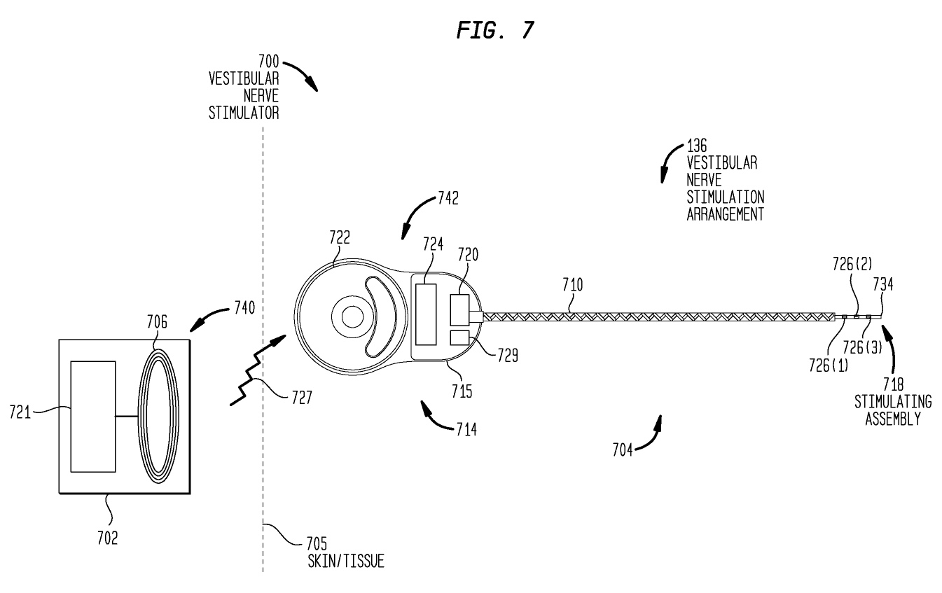

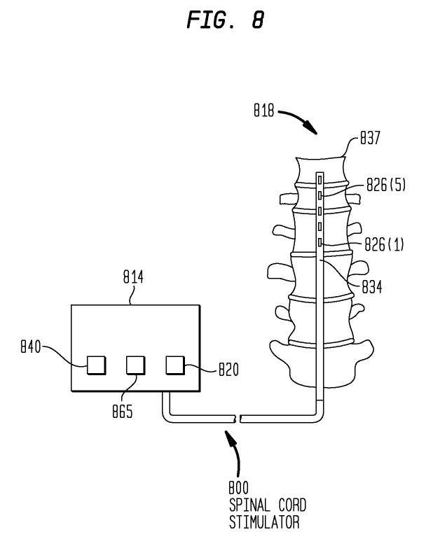

As noted above, an electrically-stimulating implantable medical device may be any medical device that is implanted in a recipient and, when implanted, is configured to deliver electrical stimulation (current) signals to the recipient. As such, the techniques presented herein may implemented with any of a number of electrically-stimulating implantable medical devices, including, for example, cochlear implants or other auditory prostheses, balance prostheses (e.g., vestibular implants), retinal or other visual prostheses, cardiac devices (e.g., implantable pacemakers), seizure devices, sleep apnea devices, electroporation devices, spinal cord stimulators, deep brain stimulators, motor cortex stimulators, sacral nerve stimulators, pudendal nerve stimulators, vagus/vagal nerve stimulators, trigeminal nerve stimulators, diaphragm (phrenic) pacers, pain relief stimulators, other neural, neuromuscular, or functional stimulators, etc. FIG. 7 is a schematic diagram illustrating a balance prosthesis with which the techniques presented herein may be implemented.

More specifically, certain individuals may suffer from a balance disorder with complete or partial loss of vestibular system function/sensation in one or both ears. In general, a balance disorder is a condition in which an individual lacks the ability to control and/or maintain a proper (balanced) body position in a comfortable manner (i.e., the recipient experiences some sensation(s) of disbalance). Disbalance, sometimes referred to herein as balance problems, can manifest in a number of different manners, such as feelings of unsteadiness or dizziness, a feeling of movement, spinning, or floating, even though standing still or lying down, falling, difficulty walking in darkness without falling, blurred or unsteady vision, inability to stand or walk un-aided, etc. Balance disorders can be caused by certain health conditions, medications, aging, infections, head injuries, problems in the inner ear, problems with brain or the heart, problems with blood circulation, etc. In general, a “balance prosthesis” or “balance implant” is a medical device that is configured to assist recipients (i.e., persons in which a balance prosthesis is implanted) that suffer from balance disorders.

As noted, FIG. 7 illustrates one example balance prosthesis, namely a vestibular nerve stimulator 700, in accordance with embodiments presented herein. More specifically, as shown in FIG. 7, the vestibular nerve stimulator 700 comprises an external component 702 and an implantable component 704, which is implantable within a recipient (i.e., implanted under the skin/tissue 705 of a recipient).

The external component 702 may comprise a number of functional and/or electronic elements used in the operation of the vestibular nerve stimulator 700. However, for ease of understanding, FIG. 7 only illustrates external radio frequency (RF) interface circuitry 721 and an external coil 706. The external coil 706 is part of an external resonant circuit 740. As described further below, the external RF interface circuitry 721 comprises data drive circuitry 744 and power drive circuitry 746 which are selectively activated/used for transcutaneous transmissions of data and power, respectively, to the implantable component 704.

The implantable component 704 comprises an implant body (main module) 714 and a vestibular stimulation arrangement 737. The implant body 734 generally comprises a hermetically-sealed housing 715 in which a number of functional and/or electronic elements used in the operation of the vestibular nerve stimulator 700 may be disposed. However, for ease of understanding, FIG. 7 only illustrates internal radio frequency (RF) interface circuitry 724, a stimulator unit 720, and a rechargeable battery 729. The implant body 734 also includes an internal/implantable coil 722 that is generally external to the housing 715, but which is connected to the internal RF interface circuitry 724 via a hermetic feedthrough (not shown in FIG. 7). The implantable coil 722 is part of an implantable resonant circuit 742. The stimulator unit 720 may include, for example, one or more current sources, switches, etc., that collectively operate to generate and deliver the electrical stimulation signals to the recipient via the vestibular stimulation arrangement 737.

As shown in FIG. 7, the vestibular stimulation arrangement 737 comprises a lead 716 and a vestibular nerve stimulating (electrode) assembly 718. The stimulating assembly 718 comprises a plurality of electrodes 726 disposed in a carrier member 734 (e.g., a flexible silicone body). In this specific example, the stimulating assembly 718 comprises three (3) electrode contacts (electrodes), referred to as electrode contacts 726(1), 726(2), and 726(3). The electrode contacts 726(1), 726(2), and 726(3) function as an electrical interface to the recipient’s vestibular nerve. It is to be appreciated that this specific embodiment with three electrodes is merely illustrative and that the techniques presented herein may be used with stimulating assemblies having different numbers of electrodes, stimulating assemblies having different lengths, etc.

The stimulating assembly 718 is configured such that a surgeon can implant the stimulating assembly, for example, adjacent the otolith organs of the peripheral vestibular system (e.g., via, the recipient’s oval window). That is, the stimulating assembly 718 has sufficient stiffness and dynamics such that the stimulating assembly can be inserted through the oval window and placed reliably within the bony labyrinth adjacent the otolith organs (e.g., sufficient stiffness to insert the stimulating assembly to the desired depth between the bony labyrinth and the membranous labyrinth).

As noted above, the external component 702 comprises an external resonant circuit 740, which includes the external coil 706. Similarly, the implantable component 704 comprises an implantable resonant circuit 742, which includes the implantable coil 722. When the coils 706 and 722 are positioned in close proximity to one another, the coils form a transcutaneous closely-coupled wireless link 727. This closely-coupled wireless link 727 formed between the external coil 706 with the implantable coil 722 may be used to transfer power and/or data from the external component 702 to the implantable component 704. That is, the external RF interface circuitry 721 is configured to drive (energize) the external coil 706 in a manner that sends power and/or data to the implantable component 704.

In the example of FIG. 7, the vestibular nerve stimulator 700 is configured to generate a localized activation field within the body of the recipient. More specifically, the vestibular nerve stimulator 700 is implanted in or adjacent to the vestibular system of the recipient and is configured to deliver electrical stimulation (current) signals to the vestibular system (e.g., peripheral vestibular system, otolith organs, vestibular nerve, etc.) of the recipient. That is, the vestibular nerve stimulator 700 sources (delivers) current to the recipient via one or more implanted electrode contacts 726(1)-726(3) or another electrode (not shown), while also sinking the current via a different one or more of the implanted electrode contacts 726(1)-726(3) or another electrode. The flow of current generated by the vestibular nerve stimulator 700 induces a localized electromagnetic field (EMF) within the immediate vicinity/proximity of vestibular nerve stimulator.

An electro-responsive biomaterial may be systemically administered to the recipient of the vestibular nerve stimulator 700, meaning the electro-responsive biomaterial is introduced into the circulatory system of the recipient (i.e., the electro-responsive biomaterial enters into systemic circulation within the body of the recipient). Since the electro-responsive biomaterial enters systemic circulation, the electro-responsive biomaterial will also flow with the oxygenated blood to tissues of the body and from the tissue capillaries back to the recipient’s heart. Stated differently, the electro-responsive biomaterial is able to circulate throughout the recipient’s body, including into the blood vessels in and/or near the recipient’s vestibular system. At this location (e.g., when the electro-responsive biomaterial flows through and/or near the vestibular system), the electro-responsive biomaterial is exposed to the localized electromagnetic field generated by vestibular nerve stimulator 700.

As noted above, the electro-responsive biomaterials in accordance with embodiments presented herein comprise a therapeutic substance and an electrically-activated carrying vehicle (electrically-activated carrier), which renders the therapeutic substance inactive/inert in the absence of a localized activation field. However, when the electro-responsive biomaterial enters a localized activation field (i.e., in response to exposure of the electro-responsive biomaterial to the localized activation field), the electrically-activated carrier reacts in a manner that enables the therapeutic substance to activate (e.g., release from its carrier, attain an activated or exposed state, etc.) to cause a therapeutic effect for the recipient at tissue in proximity to the localized activation field. In the example of FIG. 7, the localized electromagnetic field generated by vestibular nerve stimulator 700 is a localized activation field that, when the electro-responsive biomaterial is exposed thereto, a physical state of the electro-responsive biomaterial will be altered to cause a therapeutic effect to the recipient.

It is to be appreciated that the techniques presented herein have application beyond, for example, cochlear implants and balance prostheses. For example, a recipient of a cardiac device (e.g., implantable pacemaker, defibrillator, etc.), could regularly (e.g., daily) orally ingest an electro-responsive biomaterial comprising, for example, a therapeutic substance such as aspirin, a blood thinner, a nonsteroidal anti-inflammatory drug, etc. In such examples, if the cardiac device detects a cardiac event, the cardiac device could deliver electrical stimulation (e.g., generate a localized electromagnetic field) that both controls the heart and activates the therapeutic substance (e.g., aspirin) to cause some therapeutic effect for the recipient in conjunction with the cardiac event. Again, this is merely an illustrative example of the applicability of the techniques presented herein with a wide range of electrically-stimulating implantable medical devices to cause a wide range of therapeutic effects.

In another example, a recipient of a spinal cord stimulator could regularly (e.g., daily) orally ingest an electro-responsive biomaterial comprising, for example, a therapeutic substance including one or more anti-inflammatories. FIG. 8 is a simplified schematic diagram illustrating an example spinal cord stimulator 800 that maybe used in one such implementation, in accordance with embodiments presented herein.

The spinal cord stimulator 800 includes a main implantable component (implant body) 814, and a stimulating assembly 818, all implanted in a recipient. The main implantable component 814 comprises a wireless transceiver 840, a battery 865, and a stimulator unit 875. The stimulator unit 875 comprising, among other elements, one or more current sources on an integrated circuit (IC).

The stimulating assembly 818 is implanted in a recipient adjacent/proximate to the recipient’s spinal cord 837 and comprises five (5) stimulation electrodes 826, referred to as stimulation electrodes 826(1)-826(5). The stimulation electrodes 826(1)-826(5) are disposed in an electrically-insulating carrier member 834 and are electrically connected to the stimulator 820 via conductors (not shown) that extend through the carrier member 834.

Following implantation, the stimulator unit 820 is configured generate stimulation signals for delivery to the spinal cord 837 via stimulation electrodes 826(1)-826(5). Although not shown in FIG. 8, an external controller may also be provided to transmit signals through the recipient’s skin/tissue to the stimulator unit 820 for control of the stimulation signals.

In the example of FIG. 8, the spinal cord stimulator 800 is configured to generate a localized activation field within the body of the recipient. More specifically, spinal cord stimulator 800 is implanted in or adjacent to the spinal cord 837 of the recipient and is configured to deliver electrical stimulation (current) signals to the spinal cord. That is, the spinal cord stimulator 800700 sources (delivers) current to the recipient via one or more implanted electrode contacts 826(1)-826(5) or another electrode (not shown), while also sinking the current via a different one or more of the implanted electrode contacts 826(1)-826(5) or another electrode. The flow of current generated by the spinal cord stimulator 800 induces a localized electromagnetic field (EMF) within the immediate vicinity/proximity of spinal cord stimulator (i.e., the spinal cord 837). In some aspects, the electro-responsive biomaterial may migrate directionally towards the electrode.

An electro-responsive biomaterial may be systemically administered to the recipient of the spinal cord stimulator 800, meaning the electro-responsive biomaterial is introduced into the circulatory system of the recipient (i.e., the electro-responsive biomaterial enters into systemic circulation within the body of the recipient). Since the electro-responsive biomaterial enters systemic circulation, the electro-responsive biomaterial will also flow with the oxygenated blood to tissues of the body and from the tissue capillaries back to the recipient’s heart. Stated differently, the electro-responsive biomaterial is able to circulate throughout the recipient’s body, including into the blood vessels in and/or near the recipient’s spinal cord 837. At this location (e.g., when the electro-responsive biomaterial flows through and/or near the spinal cord), the electro-responsive biomaterial is exposed to the localized electromagnetic field generated by spinal cord stimulator 800.

As noted above, the electro-responsive biomaterials in accordance with embodiments presented herein comprise a therapeutic substance and an electrically-activated carrying vehicle (electrically-activated carrier), which renders the therapeutic substance inactive/inert in the absence of a localized activation field. However, when the electro-responsive biomaterial enters a localized activation field (i.e., in response to exposure of the electro-responsive biomaterial to the localized activation field), the electrically-activated carrier reacts in a manner that enables the therapeutic substance to activate (e.g., release from its carrier, attain an activated or exposed state, etc.) to cause a therapeutic effect for the recipient at tissue in proximity to the localized activation field. In the example of FIG. 8, the localized electromagnetic field generated by the spinal cord stimulator 800 is a localized activation field that, when the electro-responsive biomaterial is exposed thereto, a physical state of the electro-responsive biomaterial will be altered to cause a therapeutic effect to the recipient.

It is to be appreciated that the embodiments presented herein are not mutually exclusive.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A method, comprising:
generating, with implanted electrodes of a neurostimulation device, a localized electric field within a recipient;
in response to exposure to the localized electric field, altering a physical state of a biomaterial systematically delivered to a recipient of the neurostimulation device, wherein a change in the biomaterial, which is induced by the localized electric field, causes a release of a therapeutic substance contained within the biomaterial, and wherein the therapeutic substance is in an inactive state until after the biomaterial moves into the localized electric field; and
after release of the therapeutic substance, delivering neurostimulation to the recipient via the implanted electrodes.

2. The method of claim 1, wherein the biomaterial comprises the therapeutic substance and an electrically-activated carrier that are not covalently linked.

3. The method of claim 2, wherein the electrically-activated carrier comprises a molecular switch comprising a pocket suitable for containing the therapeutic substance, or a capsule comprising the therapeutic substance,
wherein the localized electric field causes a conformational change in the electrically-activated carrier, and
wherein the conformational change results in release of the therapeutic substance from the electrically-activated carrier.

4. The method of claim 3, wherein the electrically-activated carrier encapsulates the therapeutic substance, and further comprises a supramolecular assembly selected from the group consisting of: a liposome (lipid bilayer), a micelle (lipid monolayer), a membrane, a nanoshell, an organic nanoparticle, an inorganic nanoparticle, a dendrimer, a protein, and a fliposome.

5. The method of claim 4, wherein the liposome comprises a magnetic core.

6. The method of claim 4, wherein the liposome comprises a thermally sensitive material that degrades in a presence of the localized electric field or wherein the liposome accumulates in the presence of the localized electric field.

7. The method of claim 4, wherein the localized electric field causes one or more of a localized reduction in pH, a development of pores in an acidic environment, and a migration of the liposome to penetrate a tumor-associated tissue.

8. The method of claim 3, wherein the electrically-activated carrier encapsulates the therapeutic substance,
wherein the electrically-activated carrier further comprises a lipid layer with one or more molecules embedded in a lipid bilayer, and
wherein exposing the lipid bilayer to the localized electric field causes a conformational change resulting in a release of the therapeutic substance through an opening in the lipid layer.

9. The method of claim 1, wherein the biomaterial comprises the therapeutic substance and an electrically-activated carrier that are covalently linked.

10. The method of claim 9, wherein exposure of the biomaterial to the localized electric field, causes one or both of (i) a conformational change in the biomaterial that activates or exposes an active site of the biomaterial, and (ii) a breaking of a covalent linkage,
wherein the therapeutic substance is released.

11. The method of claim 2, wherein the localized electric field has one or more field attributes based on one or more properties of a molecular structure of the electrically-activated carrier.

12. The method of claim 1, wherein the biomaterial is delivered to the recipient via oral introduction, rectal introduction, sublingual introduction, gastric introduction, parenteral administration, intravenous introduction, intramuscular introduction, subcutaneous introduction, or transdermal introduction.

13. The method of claim 2 wherein the therapeutic substance is selected from the group consisting of: an antibiotic, an anti-inflammatory drug, an angiogenic or vasoactive agent, a growth factor, a cytotoxic agent (e.g., tumour suppressers), a biologic molecule, a nucleic acid, an antigen, and CRISPR/cas9.

14. The method of claim 1, wherein the biomaterial comprises material selected from the group consisting of: sulfonate polystyrenes, poly(thiophene)s, and/or poly(ethyloxazoline)s; polymers with ionisable groups along a backbone of a polymeric chain; naturally occurring polymers including chitosan, alginate, or hyaluronic acid; synthetic polymers that are electro-responsive including allyl amine, vinyl alcohol, acrylonitrile, methacrylic acid, or vinylacrylic acid; and both polymers that are non-electro-responsive and electro-responsive.

15. The method of claim 3, wherein the conformational change further results in the therapeutic substance being converted from the inactive state to an active state.

16. An implantable neurostimulation device, comprising:
a plurality of electrodes configured to be implanted at a target location in a recipient; and
a stimulator unit configured to:
generate, with the plurality of electrodes, a localized electric field within the recipient, wherein the localized electric field is configured to alter a physical state of a biomaterial systematically delivered to the recipient of the neurostimulation device, wherein a change in the biomaterial, which is induced by the localized electric field, causes a release of a therapeutic substance contained within the biomaterial, and wherein the therapeutic substance is in an inactive state until after the biomaterial moves into the localized electric field, and
after release of the therapeutic substance, deliver neurostimulation to the recipient via the plurality of electrodes.

17. The implantable neurostimulation device of claim 16, wherein the change is a conformational change that results in the therapeutic substance being converted from the inactive state to an active state.

18. The implantable neurostimulation device of claim 17, wherein the biomaterial further comprises an electrically-activated carrier, and wherein the conformational change causes the release of the therapeutic substance from the electrically-activated carrier.

19. The implantable neurostimulation device of claim 18, wherein the electrically-activated carrier further comprises a lipid layer with one or more molecules embedded in a lipid bilayer, and
wherein exposing the lipid bilayer to the localized electric field causes the conformational change resulting in the release of the therapeutic substance through an opening in the lipid layer.

20. The implantable neurostimulation device of claim 16, wherein the biomaterial is delivered to the recipient via oral introduction, rectal introduction, sublingual introduction, gastric introduction, parenteral administration, intravenous introduction, intramuscular introduction, subcutaneous introduction, or transdermal introduction.
